(19) United States
(12) Patent Application Publication
(10) Pub. No.: US 2024/0029849 A1
(43) Pub. Date: Jan. 25, 2024

(54) MACRO-PERSONALIZATION ENGINE FOR A VIRTUAL CARE PLATFORM

(71) Applicant: TELADOC HEALTH, INC., Purchase, NY (US)

(72) Inventor: Julia Hoffman, San Jose, CA (US)

(21) Appl. No.: 18/224,625

(22) Filed: Jul. 21, 2023

Related U.S. Application Data
(60) Provisional application No. 63/391,212, filed on Jul. 21, 2022.

(51) Int. Cl. G16H 20/00; G16H 10/20; G16H 50/30; G16H 50/70; G16H 80/00

(52) U.S. Cl. CPC G16H 20/00; G16H 10/20; G16H 50/30; G16H 50/70; G16H 80/00

(57) ABSTRACT

A computer-implemented method for personalizing a care program for a telehealth platform includes displaying a sequence of questions on a display of a user device including a plurality of personal needs questions and a plurality of questions associated with a clinical survey, receiving responses from a user to each question in the sequence of questions via an input device of the user device, storing the responses in a memory of the user device, assigning a primary concern to the user based on a response to at least a first question in the sequence of questions, assigning a severity score to the user based on the responses to the clinical survey, using a segmentation model to assign a recommended program to the user based on the primary concern and the severity score of the user, and notifying the user of the recommended program.

### CROSS-REFERENCE TO RELATED APPLICATIONS

This application claims priority to U.S. application Ser. No. 63/391,212, filed Jul. 21, 2022, the contents of which are hereby incorporated by reference in their entirety.

### TECHNICAL FIELD

The present disclosure relates generally to telehealth and/or virtual care systems and more specifically to segmenting behavioral health patients to personalize the care they receive.

BACKGROUND

Telemedicine, telehealth, and/or virtual care is the provisioning of health care services using communications devices, such as personal computers (e.g., laptops, desktops, tablets, smartphones, etc.) and/or purpose-built devices (e.g., telemedicine carts, etc.) coupled to a communications network. In many cases, virtual care involves a patient using a device to connect to and communicate with a health care provider, which may be a physician, clinician, counselor, coach, or trainer, to address health concerns of the patient. Virtual care sessions may involve a two-way audiovisual conference between the patient and the provider, communication of medical data obtained from medical instruments coupled to a communication device, communication of health records between the patient and the provider, as well as communication of a diagnosis, recommendations, and/or prescription information from the provider to the patient and/or third parties such as electronic health records providers, insurance providers, and/or pharmacies.

Today, some behavioral health services, in which patients communicate with physicians, licensed therapists, counselors, and/or coaches can be provided via virtual care platforms. Behavioral health issues are prevalent. As many as 1 in 5 people live with a diagnosable condition—more than diabetes and cancer combined. Suicide and death by overdose are increasing and have surpassed 125,000 deaths annually. In addition, behavioral health issues such as anxiety, depression, trauma, attention deficit hyperactivity disorder, diet issues, substance abuse, chronic pain, life stress, relationship stress, caregiving stress, loneliness, and occupational stress effect large portions of the population. Behavioral health issues can be linked to as much as 20% of total healthcare spending in the United States.

Many people needlessly suffer behavioral health conditions because they are unable to access care, or, if they can access care, do not receive quality care. Some barriers people face in attempting to access care include demand that outpaces supply of providers and/or facilities, insurance gaps, stigma, health literacy, logistics, cost of care, and or benefits confusion. Moreover, even when patients do access care, the quality of care can suffer for a variety of reasons, including inconsistency with patient expectations, inefficient treatment paradigms, provider burnout, overuse of emergency departments, problems in current behavioral health diagnostic strategies, lack of quality controls, and overuse of medication compared to therapy.

### SUMMARY

Virtual care platforms provide opportunities for technological solutions to the issues with behavioral healthcare described above. It would be advantageous to leverage virtual care platforms and their capabilities to improve identification and monitoring of behavioral health issues, use care replacement where appropriate, maximize provider scalability, and enhance treatment itself. These and other advantages are achieved by a macro-personalization engine in a virtual care platform. The engine assists in identifying unmet needs, segmenting populations by level of need, increasing awareness and problem recognition, as well as providing accurate, real-time tracking. The engine can also identify opportunities to expand unlicensed and/or self-management treatment options (i.e., care replacement) where appropriate. The engine functions to better map patient needs to appropriate providers, expand the reach of licensed providers, and improve the productivity of providers. Moreover, the engine can assist with ensuring adequate amounts of appropriate care, improving the quality of treatment, increasing efficiencies across venues and needs, and tracking treatment fidelity.

### BRIEF DESCRIPTION OF THE DRAWINGS

FIG. 1 is a flowchart depicting different sequences of personal needs questions (PNQs) according to an embodiment.

FIG. 2A shows examples of various levels of care acuity according to an embodiment.

FIG. 2B is a matrix for calculating a composite severity score for a segmentation model according to an embodiment.

FIG. 2C is a matrix for determining a care level for a segmentation model according to an embodiment.

FIG. 2D is a matrix for recommending a care program across a plurality of conditions according to an embodiment.

FIG. 3 is a schematic diagram of a computer system according to an embodiment.

FIG. 4 is a flowchart of a method for patient segmentation according to an embodiment.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure. Moreover, although the present disclosure discusses a macro-personalization engine in the context of behavioral health, a person skilled in the art will recognize that the systems and methods described herein can be applied to other medical conditions whose management involves regular interaction with devices, mobile apps, web portals, coaches, nurses, and/or other healthcare providers.

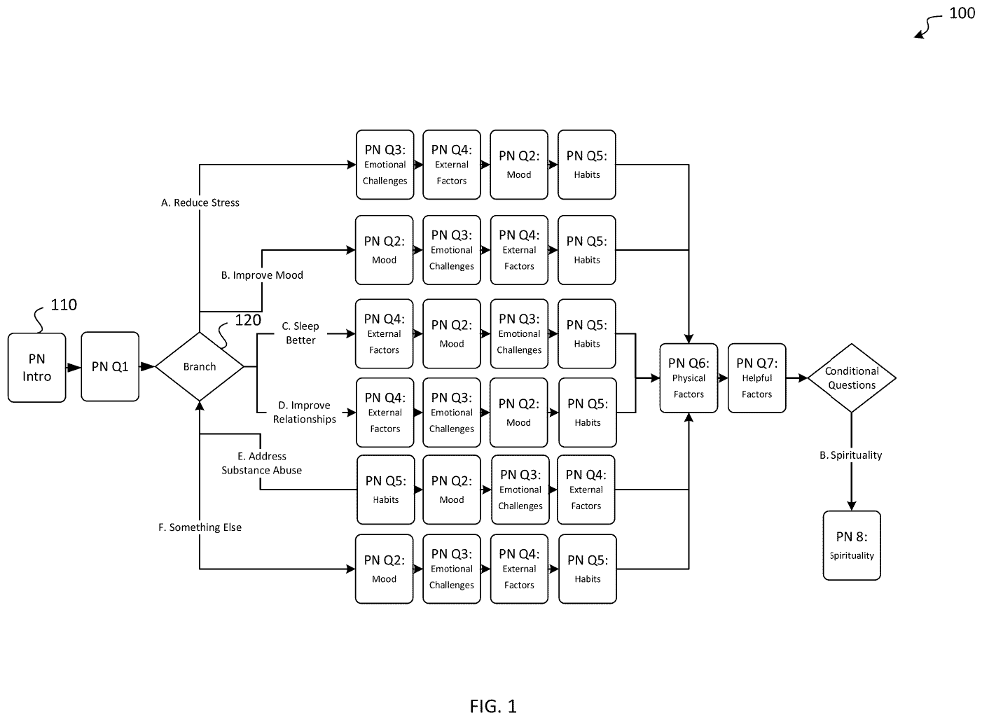

The systems and methods of the present disclosure may be implemented in a mobile application or “app” that executes on a patient device such as a smartphone or tablet, a software client executing on a desktop or laptop computer, or a web application provided by a server and displayed within a browser on any web-enabled device. The patient may first use their device to complete a registration process with a virtual care platform, such as that provided by Teladoc Health, Inc., of Purchase, New York. Once registered, the patient may be guided through a behavioral health intake process 100 depicted in FIG. 1.

After presenting the user with an introduction screen 110, the user may be presented with a sequence of personal needs questions (PNQs) designed to identify the patient's primary need or concern. By way of example, the PNQs may include those listed below in Table I.

TABLE I

| | | |
|---|---|---|
| PN Q1 | What's on your mind? | A. Stress |
| | | B. Mood |
| | | C. Sleep |
| | | D. Relationships |
| | | E. Substance use |
| | | F. Something else |
| PN Q2 | How have you been feeling | A. Angry |
| | lately? | B. Content |
| | | C. Sad |
| | | D. Hopeless |
| | | E. Worried |
| | | F. None of these |
| PN Q3 | Are you experiencing any of | A. Racing thoughts |
| | these? | B. Being impulsive |
| | | C. Intense emotions |
| | | D. Panic attacks |
| | | E. Feeling overwhelmed |
| | | F. Not feeling motivated |
| | | G. Nonsensical thoughts |
| | | H. None of these |
| PN Q4 | Are any of these causing you | A. Pregnancy or young children |
| | distress right now? | B. Caring for a loved one |
| | | C. Death of a loved one |
| | | D. Trauma |
| | | E. Work stress |
| | | F. Breakup or divorce |
| | | G. Other relationship issues |
| | | H. Other major life change |
| | | I. None of these |
| PN Q5 | Are any of these causing | A. Nicotine |
| | problems for you currently? | B. Opioids |
| | | C. Binge eating |
| | | D. Self-harm |
| | | E. Tech overuse |
| | | F. Alcohol |
| | | G. Other drugs |
| | | H. None of these |
| PN Q6 | Are any physical concerns | A. Always tired |
| | getting in the way right now? | B. Chronic pain |
| | | C. Diabetes or prediabetes |
| | | D. High blood pressure |
| | | E. Physical disability |
| | | F. Weight |
| | | G. None of these |
| PN Q7 | What do you think might | A. Mindfulness |
| | help? | B. Spirituality |
| | | C. Connecting with others |
| | | D. Medication |
| | | E. Talking to a professional |
| | | F. None of these |
| PN Q8 | What are you spiritual | A. Buddhism |
| | preferences? | B. Christianity |
| | | C. Hinduism |
| | | D. Judaism |
| | | E. Islam |
| | | F. Other religion |
| | | G. Spiritual but non-religious |

The personal needs questions may be presented to the user in different sequences depending on the user's response to PN Ql, as shown in FIG. 1. By way of example and not of limitation, if the user's answer to Q1 is “stress,” the sequence of questions might be Q3, Q4, Q2, Q5, Q6, and Q7. By contrast, if the user's answer to Q1 is “sleep,” the sequence of questions might be Q4, Q2, Q3, Q5, Q6, and Q7. In general, the sequence of the questions presented to the user will depend on the user's answer to Q1 as indicated by the branching block 120.

Once the user has completed the personal needs questionnaire, they may next be presented with one or more clinical surveys, such as the PHQ-9 or GAD-7, which are known to those skilled in the art and used to assess a person's risk for depression and anxiety. Examples of these PHQ-9 and GAD-7 clinical surveys are shown below in Tables II and III.

TABLE II
PHQ-9

| | | | | More | Nearly |
|---|---|---|---|---|---|
| Over the last 2 weeks, how often have you been bothered by | | Not | Several | than half | every |
| any of the following problems? | | at all | days | the days | day |
| #1 | Little interest or pleasure in doing things. | 0 | 1 | 2 | 3 |
| #2 | Feeling down, depressed, or hopeless. | 0 | 1 | 2 | 3 |
| #3 | Trouble falling asleep or sleeping too much. | 0 | 1 | 2 | 3 |
| #4 | Feeling tired or having little energy. | 0 | 1 | 2 | 3 |
| #5 | Poor appetite or overeating. | 0 | 1 | 2 | 3 |
| #6 | Feeling bad about yourself, or that you are a failure or | 0 | 1 | 2 | 3 |
| | have let yourself or your family down. | | | | |
| #7 | Trouble concentrating on things such as reading the | 0 | 1 | 2 | 3 |
| | newspaper or watching television. | | | | |
| #8 | Moving or speaking so slowly that other people could | 0 | 1 | 2 | 3 |
| | have noticed. Or the opposite, being so fidgety/restless | | | | |
| | that you have been moving around a lot more than usual. | | | | |
| #9 | Thoughts that you would be better off dead, or of hurting | 0 | 1 | 2 | 3 |
| | yourself in some way. | | | | |

TABLE III
GAD-7

| Over the last 2 weeks, how often | Not | Sev- | More | Nearly |
|---|---|---|---|---|
| have you been bothered by any of | at | eral | than half | every |
| the following problems? | all | days | the days | day |
| Feeling nervous, anxious, or on edge. | 0 | 1 | 2 | 3 |
| Not being able to stop or control | 0 | 1 | 2 | 3 |
| worrying. | | | | |
| Worrying too much about different | 0 | 1 | 2 | 3 |
| things. | | | | |
| Trouble relaxing. | 0 | 1 | 2 | 3 |
| Being so restless that it is hard to | 0 | 1 | 2 | 3 |
| sit still. | | | | |
| Becoming easily annoyed or irritable. | 0 | 1 | 2 | 3 |
| Feeling afraid as if something awful | 0 | 1 | 2 | 3 |
| might happen. | | | | |

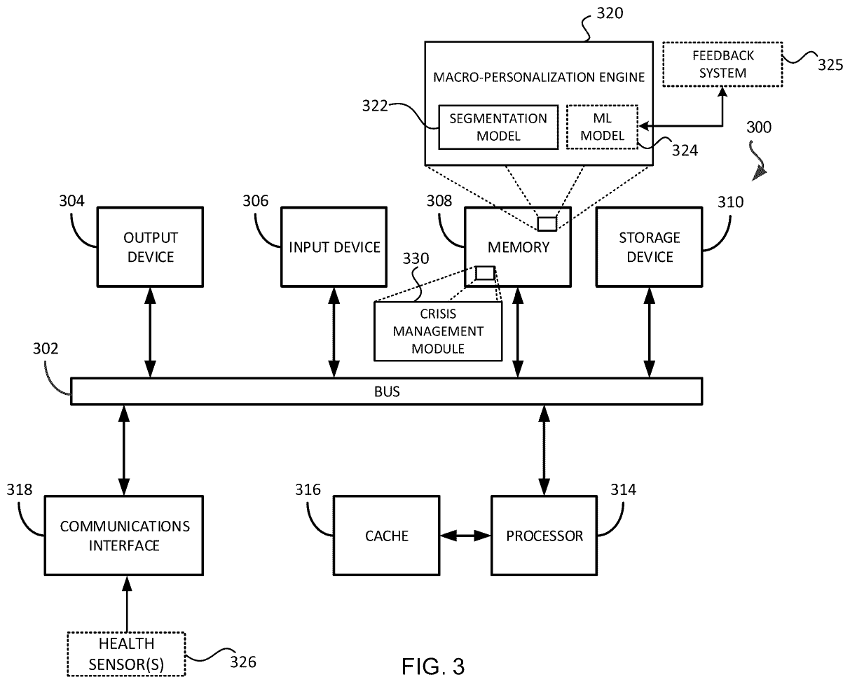

Following the user's completion of the PHQ-9 survey, if the user indicated a score of greater than 0 on PHQ-9 item #9, or if the user's overall PHQ-9 score exceeds a certain threshold (e.g., 10), the user may be directed to a crisis management module 330 (shown in FIG. 3). The crisis management module 330 may prompt the user with an option to immediately begin a consultation with a licensed therapist or other provider. The consultation may include a videoconferencing session, voice call, or text chat function.

Assuming the user's PHQ-9 responses do not trigger the crisis management module, the intake process continues and the user may be prompted to complete additional clinical surveys, such as the GAD-7 survey, a functional impairment survey, and/or an WHO-5 survey. Examples of the functional impairment survey and WHO-5 survey are shown below in Tables IV and V, respectively.

TABLE IV
Functional Impairment

| | |
|---|---|
| How difficult have these problems made it for | A. Not difficult at all |
| you to do your work, take care of things at | B. Somewhat difficult |
| home, or get along with other people? | C. Very difficult |
| | D. Extremely difficult |

TABLE V

| | | | More | Less | | |
|---|---|---|---|---|---|---|
| | All of | Most of | than half | than half | Some of | At no |
| How have you felt in the last two weeks? | the time | the time | the time | the time | the time | time |
| I have felt cheerful and in good spirits. | 5 | 4 | 3 | 2 | 1 | 0 |
| I have felt calm and relaxed. | 5 | 4 | 3 | 2 | 1 | 0 |
| I have felt active and vigorous. | 5 | 4 | 3 | 2 | 1 | 0 |
| I woke up feeling fresh and rested. | 5 | 4 | 3 | 2 | 1 | 0 |
| My daily life has been filled with things | 5 | 4 | 3 | 2 | 1 | 0 |
| that interest me. | | | | | | |

For each of the personal needs questions, the user may be prompted to choose one of the response options. For each of the clinical surveys, the user may be prompted to choose one of the numbered response options for each row, as depicted in Tables II-V.

Once the user completes all of the personal needs questions and clinical surveys they are prompted to answer, the user may be presented with a page that summarizes their results as well as a plan page that provides details on the particular care plan the system has chosen for them. The results page may display an indication to the user of the focus area determined by the engine based on the user's inputs. The plan page may include a display area including reminders to complete, for example, guided exercises that are part of the plan automatically generated for the user by the engine based on the user's inputs. The plan page may also include a display area including upcoming appointments with health care providers that have been automatically scheduled for the user by the engine based on the user's inputs.

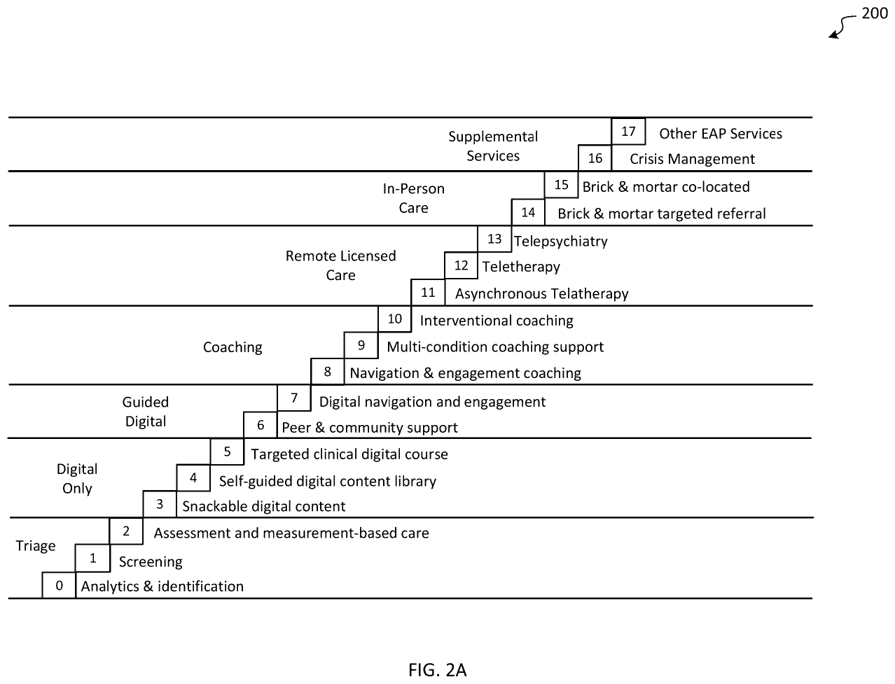

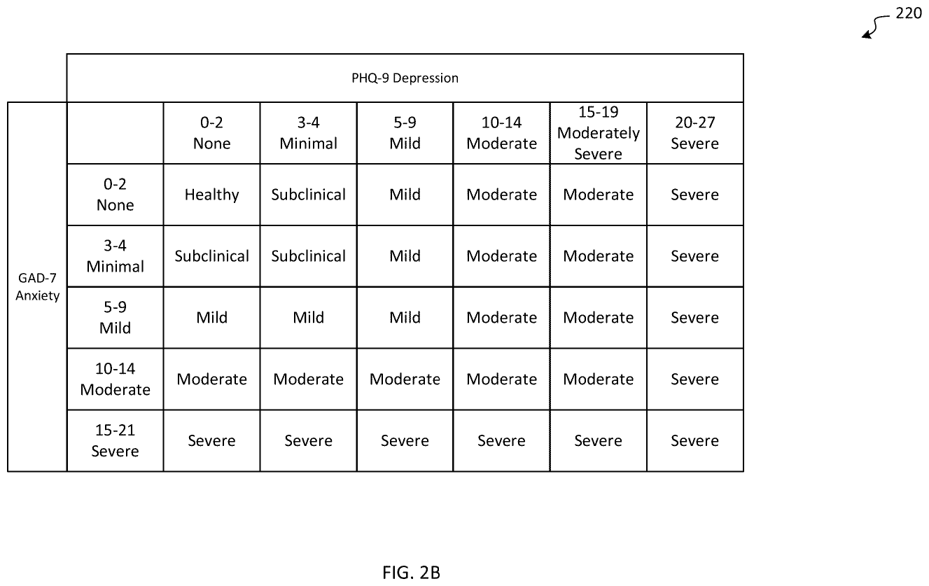

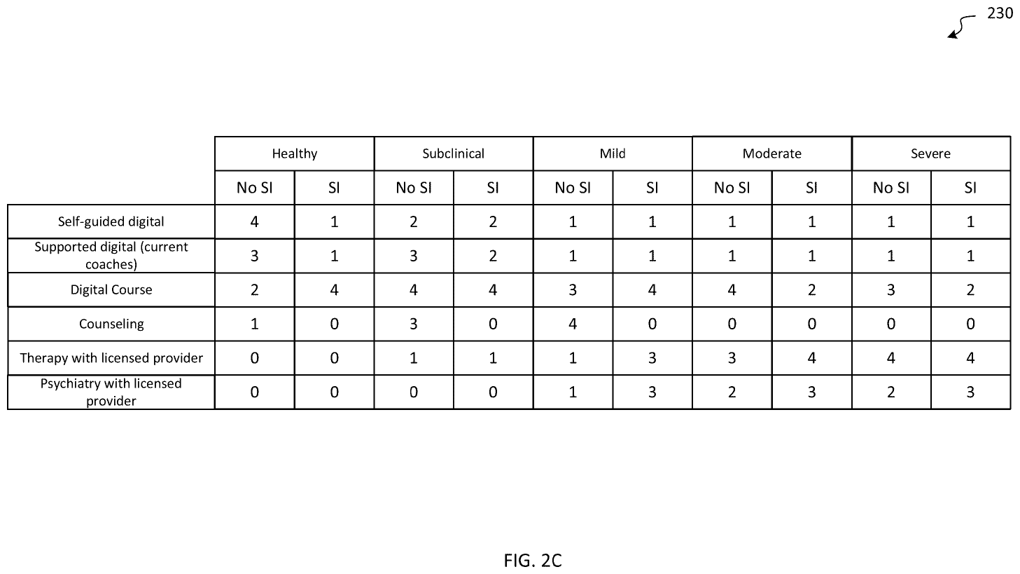

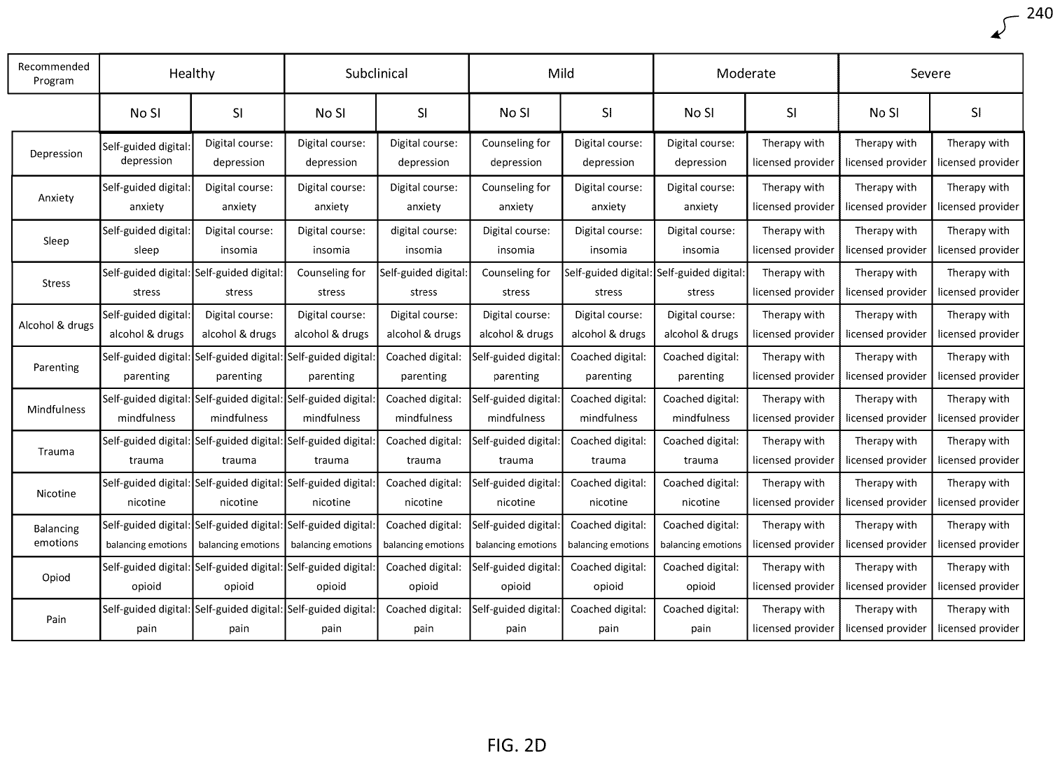

The user's responses to the personal needs questions and clinical surveys are stored and used by the mobile or web app to determine a program recommendation, suicidality risk, and a composite severity score. The app employs a model for making these determinations, as described in connection with FIGS. 2B-2D.

Once the program recommendation, suicidality, and composite severity score are determined, the user may begin the recommended program. The program may employ a tiered or stepped approach to care that provides different types of care depending on the focus area and severity of the user's condition. For low acuity conditions, the app may engage the user with an interactive, digital program module that guides the user through various techniques for addressing their primary concern. For example, for an otherwise healthy person whose primary concern is smoking cessation, the app may present the user with self-guided digital content and/or resources that include tips and techniques for quitting smoking and dealing with nicotine withdrawal. Examples of self-guided digital content for behavioral health include those included in the MYSTRENGTH product line by Livongo, Inc. For users with higher acuity conditions, the app may offer to connect the user with a live coach to provide additional level of care. And for progressively higher acuity conditions, the app may offer to connect the user with a licensed therapist or other clinician and/or trigger the crisis management module for the highest acuity conditions, such as suicidal ideation, self-harm, or similar.

In addition to monitoring and tracking users' program adherence, the app may also provide a reporting function. The reporting function may include aggregate reporting and individual reporting. For aggregate reporting, the app may compile anonymized data regarding usage of the app across all or some portion of the app's user base. This data may be useful for clinical research purposes and/or to determine which aspects of the app work as expected and which require updating. The individual reporting, on the other hand, may compile and present each user's own usage data and allow the user to visualize trends in that data over time. This may be useful for allowing the user to track their progress in the app over time.

The app may also monitor the user's adherence to the program recommendation. If the determines that the user is not keeping up with his or her recommended self-guided digital therapeutics, the app may escalate the user's condition severity and recommend live coaching sessions or therapy sessions with a licensed clinician. The app may also increase the frequency of such sessions if they are already part of the user's program recommendation. FIG. 2A shows an example of the various levels of care acuity that the app may recommend and provide based on the user's program recommendation, severity score, and other factors, such as suicidality.

FIG. 2B depicts a severity score model 220 the engine may employ to map the results of the user's clinical surveys to a severity score. Notably, even if, for example, the user produces a low score on either an anxiety assessment or a depression assessment, the user's condition may still be considered severe if they score high on the other of these two assessments.

FIG. 2C depicts an example care model 230 that can be used to select the appropriate level of care for the user based on their responses to the personal needs questions and clinical surveys. The model can be used to map the appropriateness of different care models to the severity of the user's condition. In the model 230, higher scores indicate a more appropriate mode of care for a particular severity. Lower numbers indicate a less appropriate mode of care for a particular severity. A score of zero may indicate that a particular mode of care is inappropriate for a particular severity level and the app may be configured not to offer that mode of care to the user. Otherwise, the app may be configured to one or more care modes to the user so long as each has an appropriateness score above a pre-determined threshold (e.g., 0, 1, 2, etc.). In the model 230, each severity category includes subcategories for whether or not the user indicated any suicidal ideation (“SI”). Therefore, the appropriateness of a particular mode of care can at a particular severity level can be bifurcated depending on whether the patient has indicated suicidal ideation. The appropriateness scores in model 230 are for illustrative purposes only.

FIG. 2D illustrates an example program recommendation model 240 that the app may use to automatically recommend a personalized program of care to the user based on the user's responses to the personal needs questions, clinical surveys, and using the care model 230 described above with respect to the FIG. 2C.

FIG. 3 depicts an example computer system 300 that may implement various systems and methods discussed herein. The computer system 300 may be a part of a telehealth platform that allows patients and health providers to communicate with audio and/or video over a communication network, such as the Internet. The computer system 300 includes one or more computing components in communication via a bus 302. In one implementation, the computer system 300 includes one or more processors 314. Each processor 314 may include one or more internal levels of cache 316, as well as bus controller or bus interface unit to direct interaction with a bus 302.

A memory 308 may include one or more memory cards and control circuits (not depicted), or other forms of removable memory, and may store various software applications including computer executable instructions, that when run on the processor 314, implement the methods and systems set out herein. Other forms of memory, such as a mass storage device 310, may also be included and accessible, by the processor (or processors) 314 via the bus 302.

The computer system 300 may further include a communications interface 318 by way of which the computer system 300 can connect to networks and receive data useful in executing the methods and system set out herein as well as transmitting information to other devices. The computer system 300 may include an output device 304, such as graphics card or other display interface by which information can be displayed on a computer monitor. The computer system 300 can also include an input device 306 by which information is input. Input device 306 can be a mouse, keyboard, scanner, and/or other input devices as will be apparent to a person of ordinary skill in the art.

In some embodiments, the memory 308 may include instructions to implement a macro-personalization engine 320, as described above, for segmenting patients and recommending care levels. The macro-personalization engine 320 may include or be associated with a segmentation model 322 as explained with reference to FIGS. 2A-2D. In some embodiments, the macro-personalization engine 320 may include or be associated with a trained machine learning (ML)-model 324, which may be convolutional neural network (CNN) or other suitable ML-model, for obtaining ML-derived member characteristics and/or other inferences from user responses to the sequence of questions. A feedback system 325, which may be a component of the computer system 300 or a separate system, may be used to update the ML model 324 based on feedback from a healthcare provider. For example, the ML model 324 may recommend a particular level of care. However, a reviewing healthcare provider may determine that the recommended level of care was incorrect and specify a different level of care. The feedback system 325 may then update the ML model 324 such that future recommendations are more likely to match the specified recommendations of the healthcare provider.

In some embodiments, the macro-personalization engine 320 may receive health data from one or more health sensors 326 via the communication interface 318 or another interface or mechanism. The heath sensors 326 may include, without limitation, a blood pressure sensor, a thermometer, a glucose monitor, a pulse oximeter, a camera (to capture images of the patient to identify, for example, visual signs of a stroke), or the like. The health data may be used in conjunction with the segmentation model discussed above and/or the ML model in order to segment patients and/or recommend levels of care.

The system set forth in FIG. 3 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

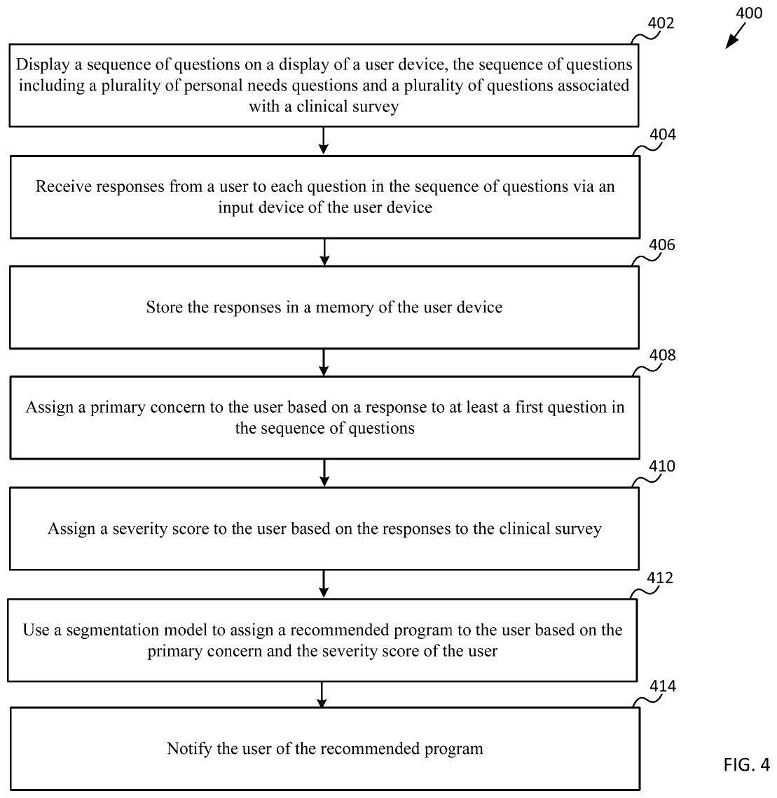

FIG. 4 is a flowchart of a computer-implemented method 400 for personalizing a care program for a telehealth platform. In one embodiment, the method 400 begins by displaying 402 a sequence of questions on a display of a user device, the sequence of questions including a plurality of personal needs questions and a plurality of questions associated with a clinical survey. The method 400 continues by receiving 404 responses from a user to each question in the sequence of questions via an input device of the user device. The method 400 then stores 406 the responses in a memory of the user device.

In some embodiments, the method 400 continues by assigning 408 a primary concern to the user based on a response to at least a first question in the sequence of questions. The method 400 may also assign 410 a severity score to the user based on the responses to the clinical survey.

Thereafter, the method 400 may use 412 a segmentation model to assign a recommended program to the user based on the primary concern and the severity score of the user and notify 414 the user of the recommended program.

The above-described disclosure may be provided as a computer program product, or software, that may include a computer-readable storage medium having stored thereon instructions, which may be used to program a computer system (or other electronic devices) to perform a process according to the present disclosure. A computer-readable storage medium includes any mechanism for storing information in a form (e.g., software, processing application) readable by a computer. The computer-readable storage medium may include, but is not limited to, optical storage medium (e.g., CD-ROM), magneto-optical storage medium, read only memory (ROM), random access memory (RAM), erasable programmable memory (e.g., EPROM and EEPROM), flash memory, or other types of medium suitable for storing electronic instructions.

The description above includes example systems, methods, techniques, instruction sequences, and/or computer program products that embody techniques of the present disclosure. However, it is understood that the described disclosure may be practiced without these specific details.

While the present disclosure has been described with references to various implementations, it will be understood that these implementations are illustrative and that the scope of the disclosure is not limited to them. Many variations, modifications, additions, and improvements are possible. More generally, implementations in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

1. A computer-implemented method for personalizing a care program for a telehealth platform, the computer-implemented method comprising:
displaying a sequence of questions on a display of a user device, the sequence of questions including a plurality of personal needs questions and a plurality of questions associated with a clinical survey;
receiving responses from a user to each question in the sequence of questions via an input device of the user device;
storing the responses in a memory of the user device;
assigning a primary concern to the user based on a response to at least a first question in the sequence of questions; and
assigning a severity score to the user based on the responses to the clinical survey;
using a segmentation model to assign a recommended program to the user based on the primary concern and the severity score of the user; and
notifying the user of the recommended program.

2. The computer-implemented method of claim 1, wherein using the segmentation model comprises using a trained machine learning (ML) model.

3. The computer-implemented method of claim 2, further comprising training the ML model based on feedback from a health care provider.

4. The computer-implemented method of claim 1, wherein using the segmentation model comprises receiving health data from one or more health sensors.

5. The computer-implemented method of claim 1, further comprising:
displaying at least one interactive page on the user device that includes self-guided content associated with the recommended program.

6. The computer-implemented method of claim 1, further comprising:
displaying a prompt on the display that includes a selectable option to begin a communication session with a health care provider; and
in response to selection by the user of the selectable option, initiating the communication session with the health care provider.

7. The computer-implemented method of claim 1, further comprising:
displaying a prompt on the display that includes a selectable option to electronically schedule an in-office appointment with a health care provider; and
in response to selection by the user of the selectable option, electronically scheduling the in-office appointment with the health care provider.

8. The computer-implemented method of claim 1, wherein displaying the sequence of questions comprises:
ordering at least a portion of the sequence of questions based on a response to at least one earlier question in the sequence of questions.

9. The computer-implemented method of claim 1, further comprising:
computing a compliance score based on adherence by the user to the recommended program; and
prompting the user to schedule a communication session with a healthcare provider when the compliance score falls below a predetermined threshold.

10. The computer-implemented method of claim 1, further comprising:
prompting the user to begin a communication session with a healthcare provider responsive to the responses of the user to the clinical survey and prior to assigning the user the recommended program.

11. A system for personalizing a care program for a telehealth platform, the system comprising:
a processor; and
a non-transitory computer-readable medium comprising program instructions that, when executed by the processor, cause the processor to:
display a sequence of questions on a display of a user device, the sequence of questions including a plurality of personal needs questions and a plurality of questions associated with a clinical survey;
receive responses from a user to each question in the sequence of questions via an input device of the user device;
store the responses in a memory of the user device;
assign a primary concern to the user based on a response to at least a first question in the sequence of questions;
assign a severity score to the user based on the responses to the clinical survey;
use a segmentation model to assign a recommended program to the user based on the primary concern and the severity score of the user; and
notify the user of the recommended program.

12. The system of claim 11, wherein the segmentation model uses a trained machine learning (ML) model.

13. The system of claim 12, further comprising a feedback system to train the ML model based on feedback from a health care provider.

14. The system of claim 11, further comprising:
a communication interface to receive health data from one or more health sensors, and wherein the segmentation model uses the health data from the one or more health sensors.

15. The system of claim 11, wherein the program instructions are to display at least one interactive page on the user device that includes self-guided content associated with the recommended program.

16. The system of claim 11, wherein the program instructions are to:
display a prompt on the display that includes a selectable option to begin a communication session with a health care provider; and
in response to selection by the user of the selectable option, initiate the communication session with the health care provider.

17. The system of claim 11, wherein the program instructions are to:
display a prompt on the display that includes a selectable option to electronically schedule an in-office appointment with a health care provider; and
in response to selection by the user of the selectable option, electronically scheduling the in-office appointment with the health care provider.

18. The system of claim 11, wherein the program instructions are to order at least a portion of the sequence of questions based on a response to at least one earlier question in the sequence of questions.

19. The system of claim 11, wherein the program instructions are to:
compute a compliance score based on adherence by the user to the recommended program; and
prompt the user to schedule a communication session with a healthcare provider when the compliance score falls below a predetermined threshold.

20. The system of claim 11, wherein the program instructions are to prompt the user to begin a communication session with a healthcare provider responsive to the responses of the user to the clinical survey and prior to assigning the user the recommended program.
